(19) United States
(12) Patent Application Publication
(10) Pub. No.: US 2009/0202452 A1
(43) Pub. Date: Aug. 13, 2009

(54) ORAL CARE REGIMEN

(75) Inventors: Richard Scott Robinson, Belle Mead, NJ (US); Richard J. Sullivan, Atlantic Highlands, NJ (US); Michael Prencipe, West Windsor, NJ (US); Rajnish Kohli, Hillsborough, NJ (US); Isabelle Van Rycke, Paris (FR); Richard Lovell, Le Vesinet (FR); Sergio Leite, Kendall Park, NJ (US); Eric A. Simon, Somerset, NJ (US); Ralph Peter Santarpia, III, Edison, NJ (US); Diane Cummins, Livingston, NJ (US); Sarita V. Mello, Somerset, NJ (US); Suman Chopra, Monroe, NJ (US); Karen J. DePierro, Piscataway, NJ (US); Lynette Zaidel, Cranford, NJ (US); Constantina Christopoulou, Somerset, NJ (US); Ravi Subramanyam, Belle Mead, NJ (US); Donghui Wu, Bridgewater, NJ (US); Andre M. Morgan, Robbinsville, NJ (US); Qin Wang, Monmouth Junction, NJ (US); Gary Edward Tambs, Belle Mead, NJ (US); Virginia Monsul Barnes, Ringoes, NJ (US)

Correspondence Address: COLGATE-PALMOLIVE COMPANY, 909 RIVER ROAD, PISCATAWAY, NJ, 08855, US

(73) Assignee: COLGATE-PALMOLIVE COMPANY, New York, NY (US)

(21) Appl. No.: 12/272,257

(22) Filed: Nov. 17, 2008

Related U.S. Application Data
(63) Continuation-in-part of application No. 12/028,782, filed on Feb. 8, 2008.
Application No. 12/028,782 is a continuation of application No. 12/058,321, filed on Mar. 28, 2008, now abandoned.
(60) Provisional application No. 61/027,431, filed on Feb. 8, 2008.
(60) Provisional application No. 61/027,420, filed on Feb. 8, 2008.
(60) Provisional application No. 61/027,435, filed on Feb. 8, 2008.
(60) Provisional application No. 61/027,432, filed on Feb. 8, 2008.

(51) Int. Cl. A61K 8/66; A61K 8/44; A61K 8/97; A61Q 11/00; A61P 31/04; A61K 8/21

(52) U.S. Cl. 424/50; 424/49; 424/52; 424/58

(57) ABSTRACT

The invention includes methods of cleaning an oral surface, maintaining oral health and/or increasing oral health. Such methods encompass contacting an oral surface with a primary oral care composition at least once daily, contacting the oral surface with a periodic oral care maintenance composition at least once monthly, wherein the periodic oral care maintenance composition comprises a basic amino acid, in free or salt form and optionally contacting the oral surface with a periodic oral care cleaning composition at least once monthly. The periodic oral care cleaning compositions suitable for use in the method comprise an abrasive system.

### CROSS REFERENCE TO RELATED APPLICATION

This application claims the benefit of U.S. Ser. No. 12/058,321, filed Mar. 28, 2008, U.S. Ser. No. 12/028,782 filed Feb. 8, 2008, U.S. Ser. No. 61/027,420 filed Feb. 8, 2008, U.S. Ser. No. 61/027,435 filed Feb. 8, 2008, U.S. Ser. No. 61/027,431 filed Feb. 8, 2008, and U.S. Ser. No. 61/027,432 filed Feb. 8, 2008 the contents of which are incorporated herein by reference.

### BACKGROUND OF THE INVENTION

Consumers report perceptions of refreshment, contentment, cleanliness, and general well-being similar to those experienced post-spa or beauty treatment after a professional teeth cleaning carried out by a dentist or hygienist. Thus, there is a need in the art for an at-home oral care regimen that permits consumers to re-create the “extra clean” mouth feel and associated sense of well-being experienced after a professional teeth cleaning and that is coupled with a regimen that preserves the dental surfaces against caries, hypersensitivity and other oral care and systemic health benefits.

### BRIEF SUMMARY OF THE INVENTION

The invention includes methods of cleaning an oral surface, maintaining oral health and/or increasing oral health. Such methods encompass contacting an oral surface with a primary oral care composition at least once daily, contacting the oral surface with a periodic oral care maintenance composition at least once monthly. The invention optionally further comprises contacting the oral surface with a periodic oral care cleaning composition at least once monthly. The periodic oral care maintenance compositions suitable for use in the method comprise a basic amino acid in free or salt form. The periodic oral care cleaning compositions suitable for use in the method comprise a first abrasive having an Einlehner hardness of greater than about 5 mg loss per 100,000 revolutions and a second abrasive having an Einlehner hardness of less than about 5 mg loss per 100,000 revolutions. In the periodic oral care cleaning compositions, the ratio of the first abrasive to the second abrasive is about 1:1.6 to about 1.6:1. The periodic oral care cleaning compositions are characterized by a pellicle cleaning ratio of greater than about 100 and a radioactive dentin abrasion of less than about 200. Additionally or alternatively, the methods may include those where the second abrasive comprises silica and has an oil of absorption of greater than about 90 cm³/100 g and/or an Einlehner hardness of less than about 5 mg loss per 100,000 revolutions. The ratio of the first abrasive to the second abrasive may be about 1:1.6 to about 1.6:1. The total amount of the first and second abrasives present in the oral composition may be greater than about 25% by weight of the composition and/or the oral composition has a pellicle cleaning ratio of greater than about 100 and a radioactive dentin abrasion of less than about 200.

The present invention provides a periodic oral care maintenance composition comprising a basic amino acid, in free or salt form, for use in the therapeutic treatment of an oral surface, which treatment comprises a. contacting the oral surface with a primary oral care composition at least once daily, and b. contacting the oral surface with the periodic oral care maintenance composition at least once monthly.

The present invention further provides a combined preparation comprising a periodic oral care maintenance composition and a primary oral care composition for the manufacture of a combined preparation for simultaneous, sequential or separate administration to a subject in the therapeutic treatment of an oral surface, wherein the oral surface is:
a. contacted with the primary oral care composition at least once daily, and
b. contacted the with the periodic oral care maintenance composition at least once monthly; and
wherein the periodic oral care maintenance composition comprises a basic amino acid, in free or salt form.

The present invention further provides the use of a basic amino acid, in free or salt form, for the manufacture of a periodic oral care maintenance composition for the therapeutic treatment of an oral surface, which treatment comprises a. contacting the oral surface with a primary oral care composition at least once daily, and b. contacting the oral surface with the periodic oral care maintenance composition at least once monthly.

The present invention further provides the use of a periodic oral care maintenance composition and a primary oral care composition for the manufacture of a combined preparation for simultaneous, sequential or separate administration to a subject in the therapeutic treatment of an oral surface, wherein the oral surface is:
a. contacted with the primary oral care composition at least once daily, and
b. contacted the with the periodic oral care maintenance composition at least once monthly; and
wherein the periodic oral care maintenance composition comprises a basic amino acid, in free or salt form.

The present invention further provides a method comprising: a. contacting an oral surface with a primary oral care composition at least once daily, and b. contacting the oral surface with a periodic oral care maintenance composition at least once monthly, wherein the periodic oral care maintenance composition comprises a basic amino acid, in free or salt form.

### DETAILED DESCRIPTION OF THE INVENTION

The invention provides a regimen method of cleaning an oral surface to remove, for example, bacteria, food particles, biofilm, plaque, and/or other agents that may result in or contribute to various oral conditions, such as bad breath, poor oral health, gingivitis, gum inflammation, or periodontitis. The regimen method of the invention also provides the user with a smooth, polished feel and appearance to the tooth surfaces that may be perceived as comparable to the “clean feel” experienced after a professional cleaning. Methods of maintaining oral health and/or increasing oral health are also provided.

As such the Compositions of the Invention are useful for treating oral conditions in the mouth, and to clean the oral cavity and provide improved methods of promoting oral health.

Enhancing oral health also provides benefits in systemic health, as the oral tissues can be gateways for systemic infections. Good oral health is associated with systemic health, including cardiovascular health. The compositions and methods of the invention provide particular benefits because basic amino acids, especially arginine, are sources of nitrogen which supply NO synthesis pathways and thus enhance microcirculation in the oral tissues. Providing a less acidic oral environment is also helpful in reducing gastric distress and creates an environment less favorable to Heliobacler, which is associated with gastric ulcers. Arginine in particular is required for high expression of specific immune cell receptors, for example T-cell receptors, so that arginine can enhance an effective immune response. The compositions and methods of the invention are thus useful to enhance systemic health, including cardiovascular health.

The primary and the periodic oral care compositions may be effective upon application to the oral cavity, e.g., with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit gingivitis, (vi) promote healing of sores or cuts in the mouth, (vii) reduce levels of acid producing bacteria, (viii) to increase relative levels of arginolytic bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) whiten teeth, (xvi) immunize the teeth against cariogenic bacteria; and/or (xvii) promote systemic health, including cardiovascular health, e.g., by reducing potential for systemic infection via the oral tissues.

The invention is an oral care regimen that includes contacting an oral surface with at least two oral care compositions at differing time increments. The oral surface to be treated includes any within the oral cavity. Exemplary surfaces include hard tissues (e.g., teeth) and soft surfaces (e.g., gingiva and tongue).

One of the oral care compositions used in the regimen method of the invention is a primary oral care composition. The primary oral care composition may be any known or to be developed in the art and can take any form. For example, the primary oral care composition may be in the form of a rinse, paste, gel, fluid, suspension, film, patch, gum, lozenge, confectionary, or semi-solid pates or stick. The primary oral care composition may be a conventional toothpaste, such as those sold under the COLGATE® trademark (Colgate-Palmolive Company, New York, N.Y.) or under the CREST® trademark (Procter & Gamble Corporation, Cincinnati, Ohio). Alternatively, the primary oral care composition may be any of those described in. e.g., U.S. Pat. Nos. 4,721,614; 4,894,220; 4,986,981; 5,037,635; 5,156,835; 5,288,480; 5,344,641; 5,538,715; 5,776,435 and United States Patent Application Publication Nos. 2004/0126332, 2004/0136924, 2005/0019273, 2005/0271601, and 2005/0271602, the contents of each of which are incorporated herein by reference.

The primary oral care composition may contain one or more agents such as humectants, solvents, thickeners, surfactants, abrasives, flavorants, colorants, viscosity and/or rheology modifiers, gums, polymers, sweeteners, etc. Examples of agents that may be present in the primary oral care composition include an antibacterial agent, a plaque dispersion agent, an antiadhesion agent, an anticaries agent, a desensitizing agent, a flavorant, a colorant, a stannous ion agent, triclosan, triclosan monophosphate, chlorhexidine, alexidine, hexetidine, sanguinarine, benzalkonium chloride, salicylanilide, domiphen bromide, cetylpyridinium chloride, tetradecylpyridinium chloride, N-tetradecyl-4-ethylpyridinium chloride, octenidine, delmopinol, octapinol, nisin, zinc ion agent, copper ion agent, essential oils, furanones, bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, and/or propolis.

In the practice of the methods of the invention, the primary oral care composition may be applied or contacted to the oral surface at least once daily. In some embodiments, the application of the primary oral care composition maybe carried out two, three, four, five, six, seven times daily, or up to fifteen times daily.

The oral surface may be contacted to the primary oral care composition using any means known or to be developed in the art; such means may vary depending on the form of the primary oral care composition. Exemplary means of contacting the primary oral care composition to the oral surface include application using an implement (such as a brush, toothbrush, stick, sponge, cotton swab), lavage, chewing, adjacent placement, and dissolution of confectionary.

Another of the oral care compositions used in the methods of the invention is a periodic oral care maintenance composition. The oral care maintenance composition is generally applied by the consumer. The oral care maintenance composition is in a form selected from a mouthrinse, a toothpaste, a tooth gel, a tooth powder, a non-abrasive gel, a mousse, a foam, a mouth spray, a lozenge, an oral tablet, a varnish, or a sealant. The periodic oral care maintenance composition comprises an effective amount of a basic amino acid, in free or salt form, e.g., arginine. The composition may further comprise an effective amount of an antibacterial agent, e.g., triclosan and/or Zn⁺² ion source, e.g., zinc citrate: an effective amount of a fluoride source, e.g., a soluble fluoride salt; and/or a polymer.

In particular, the antibacterial agent may be selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanidc antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, zinc citrate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.

In some embodiments, the application of the oral care maintenance composition may be carried out daily, every other day, weekly, monthly or any other interval based on the oral care maintenance composition delivery form and desired end benefit. For example, when the oral care delivery form is a toothpaste the use is e.g., at least daily, when the oral care delivery form is a varnish the use is e.g., weekly or monthly.

Another of the oral care composition used in the methods of the invention is a periodic oral care cleaning composition. The periodic oral care cleaning composition typically has a Pellicle Cleaning Ratio (“PCR”) of greater than about 100, while having a Radiotracer Dentin Abrasion (“RDA”) of less than about 200. In certain embodiments, the RDA is less than or equal to about 175, while still having a PCR that exceeds about 100. In some embodiments, the RDA is less than about 165. (Methods of performing PCR and RDA are described in e.g., U.S. Pat. Nos. 5,939,051 and 6,290,933, both of which are herein incorporated by reference in their entireties.)

The periodic oral care cleaning composition may include a first abrasive and a second abrasive; such abrasives are described in United States Publication No. 2007/0140986, the contents of which are incorporated herein by reference.

In certain embodiments, the periodic oral composition comprises a first abrasive having an Einlehner hardness of greater than about 5 mg loss per 100,000 revolutions and a second abrasive having an Einlehner hardness of less than about 5 mg loss per 100,000revolutions. The first particle having an Einlehner hardness of greater than about 5 mg loss per 100,000 revolutions has a primary a polishing function when it is contacted with an oral surface. The second particle having an Einlehner hardness of less than about 5 mg loss per 100,000 revolutions has a primary cleaning function, as it is a softer particle. In certain embodiments, the second particle has a hardness of greater than about 10 mg loss per 100,000 revolutions, and in other embodiments, greater than about 15 mg loss per 100,000 revolutions.

The first abrasive of the periodic oral care cleaning composition may be of a cleaning abrasive that has a hardness of less than or equal to the oral surface to be treated, and the second abrasive is a polishing abrasive that has a hardness of greater than or equal to the oral surface to be treated.

Each of the first abrasive and second abrasive of the periodic oral care cleaning composition has a particle size. In certain embodiments, the first particle size is less than the second particle size. Mean particle size can be measured, e.g., using a Malvern Particle Size Analyzer, Model Mastersizer S. Malvern Instruments, Inc. of Southborough, Mass. U.S.A.

In certain embodiments, the first abrasive has a mean particle size of less than about 11 μm, e.g., less than about 10 μm. For example, examples of suitable abrasives have mean particle sizes ranging from about 7 μm to about 11 μm. Some abrasives have particle sizes of less than about 5 μm. In other embodiments, the second abrasive has a mean particle size of greater than about 8 μm, e.g., greater than about 10 μm. In some embodiments, the second abrasive can have a mean particle size of about 8 μm to about 14 μm.

Any orally or cosmetically acceptable abrasive fulfilling the requirements set forth above can be selected for use in the periodic oral care cleaning composition. Suitable abrasives include without limitation, silica, silicate, silicon, alumina (including calcined aluminum oxide), aluminosilicates, such as bentonite, zeolite, kaolin, and mica, siliceous or diatomaceous earth, pumice, calcium carbonate, cuttlebone, insoluble phosphates, composite resins, such as melamine resin, phenolic resin, and urea-formaldehyde resin, polycarbonate, silicon carbide, boron carbide, microcrystalline wax, microcrystalline cellulose, including combinations of colloidal microcrystalline cellulose and carboxymethylcellulose, commercially available under the trade name AVICEL® from FMC Biopolymer of Philadelphia, Pa. U.S.A. and combinations and derivatives of all of the above. By “mica” it is meant any one of a group of hydrous aluminum silicate minerals with plate morphology and perfect basal (micaceous) cleavage. Mica can be, for example, sheet mica, scrap mica or flake mica, as exemplified by muscovite, biotite or phlogopite type micas. Among insoluble phosphates useful as abrasives are orthophosphates, polymetaphosphates and pyrophosphates. Illustrative examples are dicalcium orthophosphate dihydrate, dicalcium phosphate dihydrate, calcium hydrogen phosphate, calcium pyrophosphate, β-calcium pyrophosphate, tricalcium phosphate, calcium metaphosphate, potassium metaphosphate, and sodium metaphosphate.

Synthetic silicas include both silica gels and precipitated silicas which may be prepared, for example, by the neutralization of aqueous silicate solutions with a strong mineral acid. In the preparation of silica gel, a silica hydrogel is formed which is then typically washed to low salt content. The washed hydrogel may be milled to the desired size, or otherwise dried, ultimately to the point where its structure no longer changes as a result of shrinkage. When preparing such synthetic silicas, the objective is to obtain abrasives which provide maximal cleaning (i.e., removal of stained pellicle) with minimal damage to the tooth enamel and other oral tissue.

Useful abrasive materials for preparing the periodic oral compositions include high cleaning, low structure silica abrasives, such as those marketed under the trade designation SYLODENT® XWA or SYLODENT® 783 by Davison Chemical Division of W. R. Grace & Co. of Baltimore, Md. SYLODENT® XWA 650 is a silica hydrogel composed of particles of colloidal silica. Exemplary silica hydrogels comprise colloidal particles of silica having an average particle size of about 3 μm to about 12 μm, and e.g., between about 5 μm to about 10 μm, with a pH range from about 4 to about 10, e.g., about 6 to about 9 when measured as a 5% by weight slurry. The particles of the XWA 650 contain about 10% to about 35% by weight water, have a mean particle size of about 5 μm to about 12 μm, an Einlehner hardness of from greater than or equal to about 5 to about 20 mg loss per 100,000 revolutions, an oil absorption of less than about 90 cm³/100 g, for example from between about 40 cm³/100 g to about 90 cm³/100 g. The abrasives have a Brunauer, Emmett and Teller (BET) surface area from about 100 to about 700 m²/g. XWA 650 has a brightness of about 96.8 technidyne. Such abrasives are disclosed in, e.g., U.S. Pat. No. 6,290,933, which is incorporated herein by reference in its entirety.

Another high cleaning silica abrasive that can be included in the periodic composition is marketed as SYLODENT® XWA 300 and is a silica hydrogel containing about 10% to about 25% water by weight, where the mean particles size is about 2 μm to about 4 μm. The particles have BET surface area in the range of about 150 to about 400 m²/g of silica. The XWA 300 abrasive has an oil absorption of less than about 90 cm³/100 g silica: and a pH, in a 5% by weight suspension in boiled (CO₂ free) demineralized water, equal to or greater than about 8.5. Such abrasives are disclosed in U.S. Pat. No. 5,939,051, which is incorporated herein by reference in its entirety.

Another suitable high cleaning silica that can be included in the periodic composition comprises a silica product, where the particles are about 5% to about 35% by weight water, having a mean particle size of about 7 μm to about 11 μm, an Einlehner hardness of from about 12 to about 19, an oil absorption value of about 50 cm³/100 g to about 65 cm³/100 g. A BET surface area is about 100 to about 700 m²/g of silica. The brightness is generally reported to be greater than about 95 technidyne. Such a silica product is commercially available as ZEODENT® 105 from J. M. Huber of Havre de Grace, Md., U.S.A.

Other abrasives that can be used include typical cleaning silica abrasives, such as precipitated silicas having a mean particle size of up to about 20 μm, typically at about 8 to about 14 μm, with an oil absorption structure of greater than about 90 to about 110 cm³/100 g, such as ZEODENT® 115, marketed by J. M. Huber, that has a pH at 5% of the particles of about 6.5-about 7.5 and an Einlehner hardness of about 2 to about 4 mg loss per 100,000 revolutions. The brightness of such a silica particle is greater than about 95. Such cleaning abrasives comprise the second abrasive of the oral composition in certain embodiments.

The silica abrasive polishing materials useful herein, as well as the other abrasives, generally have an average particle size ranging between about 0.1 and about 30 microns, about between 5 and about 15 microns. The silica abrasives can be from precipitated silica or silica gels, such as the silica xerogels described in U.S. Pat. No. 3,538,230, to Pader et al. and U.S. Pat. No. 3,862,307, to Digiulio, both incorporated herein by reference. Particular silica xerogels are marketed under the trade name Syloid® by the W. R. Grace & Co., Davison Chemical Division. The precipitated silica materials include those marketed by the J. M. Huber Corp. under the trade name Zeodent®, including the silica carrying the designation Zeodent 115 and 119. These silica abrasives are described in U.S. Pat. No. 4,340,583, to Wason, incorporated herein by reference.

In certain embodiments, abrasive materials useful in the practice of the oral care compositions in accordance with the invention include silica gels and precipitated amorphous silica having an oil absorption value of about less than 100 cc/100 g silica and in the range of about 45 cc/100 g to about 70 cc/100 g silica. Oil absorption values are measured using the ASTA Rub-Out Method D281. In certain embodiments, the silicas are colloidal particles having an average particle size of about 3 microns to about 12 microns, and about 5 to about 10 microns.

In particular embodiments, the particulate or abrasive materials comprise a large fraction of very small particles, e.g., having a d50 less than about 5 microns, for example small particle silica (SPS) having a d50 of about 3 to about 4 microns, for example Sorbosil AC43® (Ineos). Such small particles are particularly useful in formulations targeted at reducing hypersensitivity. The small particle component may be present in combination with a second larger particle abrasive. In certain embodiments, for example, the formulation comprises about 3 to about 8% SPS and about 25 to about 45% of a conventional abrasive.

Low oil absorption silica abrasives particularly useful in the practice of the invention are marketed under the trade designation Sylodent XWA® by Davison Chemical Division of W.R. Grace & Co., Baltimore, Md. 21203, Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive useful in the practice of the present invention. The abrasive is present in the oral care composition of the present invention at a concentration of about 10 to about 60% by weight, in other embodiment about 20 to about 45% by weight, and in another embodiment about 30 to about 50% by weight.

In embodiments where the dentifrice is a clear or transparent gel, an abrasive of colloidal silica, such as those sold under the trademark SYLOID® as SYLOID® 72 and SYLOID® 74 or under the trademark SANTOCEL® 100 alkali metal alumina-silicate complexes are particularly useful, since they have refractive indices close to the refractive indices of gelling agent-liquid (including water and/or humectant) systems commonly used in dentifrices.

In accordance with various embodiments of the present invention, a first and a second abrasive are combined in the periodic composition to provide cleaning capability, mildness (relatively low abrasivity), and aesthetically acceptable periodic care compositions. In various embodiments, an amount of the first abrasive to the second abrasive can be expressed by a ratio of the first abrasive to the second abrasive ranging from about 1:1.6 to about 1.6:1. For example, a ratio of the first abrasive to the second abrasive is about 1:1. The respective amounts of the first abrasive and the second abrasive present in an oral composition may be about 13 to about 21% by weight of the oral composition.

In some embodiments, the amount of the first abrasive is about 15% to about 19%, and the amount of the second abrasive is about 15% to about 19% by weight of the oral composition. In certain preferred embodiments, the first abrasive is present at about 17%) by weight and the second abrasive is present at about 17% by weight of the oral composition. Thus, in accordance with various embodiments of the present invention, the total amount of abrasive in the periodic oral care cleaning composition, including the first and second abrasive is e.g., greater than about 25%, greater than about 30%, and in some embodiments, greater than about 35% by total weight of the periodic oral care cleaning composition.

In some embodiments, the periodic oral composition comprises a first abrasive having an Einlehner hardness of greater than about 5 mg loss per 100,000 revolutions and a second abrasive having an Einlehner hardness of less than about 5 mg loss per 100,000 revolutions. A ratio of the first abrasive to the second abrasive ranges from about 1:1.6 to about 1.6:1. Further, the periodic oral composition has a PCR of greater than about 100 and an RDA of less than about 200. In certain embodiments, the RDA is less than about 175. In certain embodiments, the first abrasive has an oil of absorption of less than about 90 cm³/100 g, and the second abrasive has an oil of absorption of greater than about 90 cm³/100 g. In some embodiments, the ratio of the first abrasive to the second abrasive is about 1:1. The total amount of the abrasives, including both the first and the second abrasives, is greater than about 30%. The first abrasive and the second abrasive are optionally present at about 17%) each, respectively. In some embodiments, the first and second abrasives are respectively present in an amount of about 15% to about 19% by weight of the total periodic oral care cleaning composition. The first and second abrasives optionally comprise silica.

In certain embodiments, the periodic oral composition comprises a first abrasive comprising silica, having an Einlehner hardness of greater than about 5 mg loss per 100,000revolutions, and an oil of absorption of less than about 90 cm³/100 g. The periodic oral composition further comprises a second abrasive comprising silica having an Einlehner hardness of less than about 5 mg loss per 100,000 revolutions, and an oil of absorption of greater than about 90 cm³/100 g. The first abrasive is present at an amount of about 13% to about 21%) by weight, likewise, the second abrasive is present in an amount of about 13% to about 21% by weight of the composition. In some embodiments, the first abrasive and the second abrasive are respectively present at about 15% to about 19% by weight of the periodic oral composition. In certain embodiments, the first and second abrasives are respectively present in an amount of about 17% by weight of the oral composition. The PCR is e.g.. greater than about 100 and the RDA is e.g., less than about 200, and in certain embodiments, less than about 175. A total amount of the first and second abrasive present in the oral composition are e.g., greater than about 25% or e.g., greater than about 30%.

In certain embodiments, the periodic oral composition comprises a first abrasive comprising silica and having an oil of absorption of less than about 90 cm³/100 g and an Einlehner hardness of greater than about 5 mg loss per 100,000 revolutions. The periodic oral composition further comprises a second abrasive comprising silica and having an oil of absorption of greater than about 90 cm³/100 g and an Einlehner hardness of less than about 5 mg loss per 100,000 revolutions. A ratio of the first abrasive to the second abrasive is about 1:1.6 to about 1.6:1. Further, a total amount of the first and second abrasives present in the oral composition is greater than about 25% by weight of the composition.

In certain embodiments, the periodic oral composition has a ratio of the first abrasive to the second abrasive that ranges from about 1:1.6 to about 1.6:1. In particular embodiments, the active ingredient comprises a non-ionic antibacterial ingredient, such as a halogenated diphenyl ether like triclosan, which will be discussed in more detail below. In some embodiments, the first abrasive has an oil of absorption of less than about 90 cm³/100 g and an Einlehner hardness of greater than about 5 mg loss per 100,000 revolutions, and the second abrasive has an oil of absorption of greater than about 90 cm³/100 g and an Einlehner hardness of less than about 5 mg loss per 100,000 revolutions.

The periodic oral care cleaning compositions described herein (both the primary and the periodic) may comprise an orally acceptable carrier. Conventional ingredients that can be used to form the carriers for oral care compositions are well known to the skilled artisan. The carrier can be a liquid, semi-solid, or solid phase. Oral compositions can be in the form of a dentifrice (including toothpastes, toothpowders, and prophylaxis pastes), confectionaries (including gums, beads and chews), film, paint-on gels, or any other form known to one of skill in the art where abrasives are employed. Selection of specific carrier components is dependant on the desired product form.

In certain preferred embodiments, the composition(s) are independently in the form of a dentifrice, where an exemplary carrier is substantially semi-solid or solid. The carrier can be aqueous, in which case the carrier e.g., comprises about 5% to about 95% water. In other embodiments, the carrier is substantially non-aqueous. The carrier optionally comprises, for example, oral care active ingredients, surface active agents, such as surfactants, emulsifiers, and foam modulators, viscosity modifiers and thickeners, humectants, diluents, fillers, additional pH modifying agents, colorants, preservatives, solvents, and combinations thereof. It is understood that while general attributes of each of the above categories of materials may differ; there may be some common attributes and any given material may serve multiple purposes within two or more of such categories of materials. As recognized by one of skill in the art, the oral composition(s) optionally include other materials in addition to those components previously described, including for example, emollients, moisturizers, mouth feel agents and the like. Examples of suitable carriers for oral compositions are disclosed in U.S. Pat. Nos. 6,669,929, 6,379,654, and 4,894,220, the content of each of which are incorporated herein by reference.

The oral care active ingredients include for example, anti-bacterial active agents, anti-tartar agents, anti-caries agents, anti-inflammatory agents, anti-sensitivity agents, enzymes, nutrients, and the like. Active agents among those useful herein are also disclosed in U.S. Pat. Nos. 6,290,933 and 6,685,921, the contents of each of which are incorporated herein by reference.

The primary and/or periodic oral care compositions may further comprise an agent that interferes with or prevents bacterial attachment, e.g., solbrol or chitosan.

Any suitable fluoride ion source may be present in either or both of the oral composition(s), such as those recited in U.S. Pat. No. 5,080,887, the contents of which are incorporated herein by reference. Sources of fluoride ions, acid phosphatases, and pyrophosphatase enzyme inhibitors, are well known in the art as anti-caries agents. Examples of such sources are inorganic metal and/or ammonium fluoride salts and compounds, such as, for example: sodium fluoride, potassium fluoride, ammonium fluoride, amine fluoride, calcium fluoride; a copper fluoride, such as cuprous fluoride; zinc fluoride, barium fluoride; sodium silicafluoride, sodium fluorosilicate, ammonium fluorosilicate, sodium fluorozirconate; and sodium monofluorophosphate, aluminum mono- and di-fluorophosphate, and fluorinated sodium calcium pyrophosphate, and combinations thereof. The fluoride source can also be an amine fluoride, such as olaflur (N′octadecyltrimethylendiamine-N,N,N′-tris(2-ethanol)-dihydrofluoride). Sodium fluoride, amine fluoride, stannous fluoride, sodium monofluorophosphate (MFP), and mixtures thereof, are preferred. In certain embodiments the fluoride ion source includes stannous fluoride, sodium fluoride, sodium monofluorophosphate as well as mixtures thereof.

In certain embodiments, the periodic and/or primary oral care compositions of the invention may also contain a source of fluoride ions or fluorine-providing ingredient in amounts sufficient to supply about 25 ppm to 25,000 ppm of fluoride ions, generally at least about 500 ppm, e.g., about 500 to about 2000 ppm, e.g., about 1000 to about 1600 ppm, e.g., about 1450 ppm. The appropriate level of fluoride will depend on the particular application. A mouthwash, for example, would typically have about 100 to about 250 ppm fluoride. A toothpaste for general consumer use would typically have about 1000 to about 1500 ppm, with pediatric toothpaste having somewhat less. A dentifrice or coating for professional application could have as much as 5,000 or even 25,000 ppm fluoride.

The periodic and/or primary oral care compositions of the invention may comprise a calcium phosphate abrasive, e.g.. tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate.

Either or both oral composition(s) optionally comprise an anticalculus component, such as one or more of the anti-calculus components recited in U.S. Pat. No. 5,292,526, the contents of which are incorporated herein by reference. In various embodiments, the anti-calculus component includes one or more polyphosphates. The anti-calculus composition can include at least one wholly or partially neutralized alkali metal or ammonium tripolyphosphate or hexametaphosphate salt present in the oral composition at an effective anti-calculus amount. The anti-calculus component can also include at least one water soluble, linear, molecularly dehydrated polyphosphate salt effective in an anticalculus amount. The anti-calculus component can also include a mixture of potassium and sodium salts, at least one of which is present in an effective anti-calculus amount as a polyphosphate anti-calculus agent. Other useful anticalculus agents include polycarboxylate polymers and polyvinyl methyl ether/maleic anhydride (PVM/MA) copolymers, such as GANTREZ®.

All other substances or molecules known to be useful in oral care compositions may be included in either or both of the daily or the periodic compositions. For example, such additional molecules may include antibacterial agents, antiplaque agents, desensitizing agents, anti-inflammation agents, colorants, thickeners, flavorants, surfactants, abrasives, anti-adhesion agents, an anti-caries agent, a sensate, and/or vitamins.

Other components that may be added to either or both composition include a stannous ion agent: triclosan; triclosan monophosphate; chlorhexidine; alexidine; hexetidine; sanguinarine; benzalkonium chloride; salicylanilide; domiphen bromide: cetylpyridinium chloride (CPC); tetradecylpyridinium chloride (TPC): N-tetradecyl-4-ethylpyridinium chloride (TDEPC); octenidine; delmopinol; octapinol; nisin; zinc ion agent; copper ion agent; essential oils: furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, chitosan and propolis.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

We claim:

1. A periodic oral care maintenance composition comprising a basic amino acid, in free or salt form, for use in the therapeutic treatment of an oral surface, which treatment comprises a. contacting the oral surface with a primary oral care composition at least once daily, and b. contacting the oral surface with the periodic oral care maintenance composition at least once monthly.

2. The periodic oral care maintenance composition of claim 1 further comprising at least one of an effective amount of an antibacterial agent, an effective amount of a fluoride source, and/or a polymer.

3. The periodic oral care maintenance composition of claim 1, wherein the basic amino acid comprises arginine.

4. The periodic oral care maintenance composition of claim 3 wherein the basic amino acid is in salt form and selected from arginine phosphate, arginine bicarbonate, and arginine hydrochloride.

5. A combined preparation comprising a periodic oral care maintenance composition and a primary oral care composition for the manufacture of a combined preparation for simultaneous, sequential or separate administration to a subject in the therapeutic treatment of an oral surface, wherein the oral surface is:
a. contacted with the primary oral care composition at least once daily, and
b. contacted the with the periodic oral care maintenance composition at least once monthly; and
wherein the periodic oral care maintenance composition comprises a basic amino acid, in free or salt form.

6. The combined preparation of claim 5 wherein the periodic oral care maintenance composition further comprises at least one of an effective amount of an antibacterial agent, an effective amount of a fluoride source, and/or a polymer.

7. The combined preparation of claim 5, wherein the basic amino acid comprises arginine.

8. The combined preparation of claim 7 wherein the basic amino acid is in salt form and selected from arginine phosphate, arginine bicarbonate, and arginine hydrochloride.

9. The combined preparation of claim 5, wherein the primary oral care composition comprises an agent selected from an abrasive agent, an antibacterial agent, a plaque dispersion agent, an antiadhesion agent, an anticaries agent, a desensitizing agent, a flavorant, a colorant, and a sensate.

10. The combined preparation of claim 5, wherein the primary oral care composition comprises an agent selected from a stannous ion agent; triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, chitosan and propolis.

11. Use of a basic amino acid, in free or salt form, for the manufacture of a periodic oral care maintenance composition for the therapeutic treatment of an oral surface, which treatment comprises a. contacting the oral surface with a primary oral care composition at least once daily, and b. contacting the oral surface with the periodic oral care maintenance composition at least once monthly.

12. The use of claim 11, wherein the basic amino acid comprises arginine.

13. The use of claim 12 wherein the basic amino acid is in salt form and selected from arginine phosphate, arginine bicarbonate, and arginine hydrochloride.

14. Use of a periodic oral care maintenance composition and a primary oral care composition for the manufacture of a combined preparation for simultaneous, sequential or separate administration to a subject in the therapeutic treatment of an oral surface, wherein the oral surface is:
a. contacted with the primary oral care composition at least once daily, and
b. contacted the with the periodic oral care maintenance composition at least once monthly; and
wherein the periodic oral care maintenance composition comprises a basic amino acid, in free or salt form.

15. The use of claim 14 wherein the periodic oral care maintenance composition further comprises at least one of an effective amount of an antibacterial agent, an effective amount of a fluoride source, and/or a polymer.

16. The use of claim 14, wherein the basic amino acid comprises arginine.

17. The use of claim 16 wherein the basic amino acid is in salt form and selected from arginine phosphate, arginine bicarbonate, and arginine hydrochloride.

18. The use of claim 14, wherein the primary oral care composition comprises an agent selected from an abrasive agent, an antibacterial agent, a plaque dispersion agent, an antiadhesion agent, an anticaries agent, a desensitizing agent, a flavorant, a colorant, and a sensate.

19. The use of claim 14, wherein the primary oral care composition comprises an agent selected from a stannous ion agent; triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, chitosan and propolis.

20. A method comprising:
a. contacting an oral surface with a primary oral care composition at least once daily, and
b. contacting the oral surface with a periodic oral care maintenance composition at least once monthly, wherein the periodic oral care maintenance composition comprises a basic amino acid, in free or salt form.

21. The method of claim 20 further comprising at least one of an effective amount of an antibacterial agent, an effective amount of a fluoride source, and/or a polymer.

22. The method of claim 20, wherein the basic amino acid comprises arginine.

23. The method of claim 22 wherein the basic amino acid is in salt form and selected from arginine phosphate, arginine bicarbonate, and arginine hydrochloride.

24. The method of claim 20 further comprising contacting the oral surface with a periodic oral care cleaning composition at least once monthly.

25. The method of claim 20, wherein the primary oral care composition comprises an agent selected from an abrasive agent, an antibacterial agent, a plaque dispersion agent, an antiadhesion agent, an anticaries agent, a desensitizing agent, a flavorant, a colorant, and a sensate.

26. The method of claim 20, wherein the primary oral care composition comprises an agent selected from a stannous ion agent: triclosan; triclosan monophosphate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; delmopinol; octapinol; nisin; zinc ion agent; copper ion agent; essential oils; furanones; bacteriocins, ethyl lauroyl arginate, extracts of magnolia, a metal ion source, arginine bicarbonate, honokiol, magonol, ursolic acid, ursic acid, morin, extract of sea buckthorn, a peroxide, an enzyme, a Camellia extract, a flavonoid, a flavan, halogenated diphenyl ether, creatine, chitosan and propolis.

27. The method of claim 20, wherein the primary oral care composition is applied two to five times daily.

28. The method of claim 20, wherein the oral surface is contacted with the periodic oral care maintenance composition at least weekly.

29. The method of claim 20, wherein the oral surface is contacted with the periodic oral care maintenance composition at least daily.
